(19) United States
(12) Patent Application Publication
(10) Pub. No.: US 2026/0248649 A1
(43) Pub. Date: Aug. 27, 2026

(54) SURGICAL LASER SYSTEM INTEGRITY VERIFICATION

(71) Applicant: Alcon Inc., Fribourg (CH)

(72) Inventors: David Jung, Tustin, CA (US); John Hossein Karim, Cypress, CA (US)

(21) Appl. No.: 19/457,398

(22) Filed: Jan. 23, 2026

Related U.S. Application Data
(60) Provisional application No. 63/763,380, filed on Feb. 26, 2025.

(51) Int. Cl. A61F 9/008

(52) U.S. Cl. CPC A61F 9/008

(57) ABSTRACT

In certain embodiments, a surgical laser system includes a laser probe and a laser subsystem. The laser probe is configured to emit a treatment light beam and an illumination light beam and receive a back-reflected illumination light beam. The laser subsystem includes a pulsed laser source, a continuous wave laser source, an optical sensor, and a controller. The pulsed laser source is configured to generate the treatment light beam in response to a control signal. The continuous wave laser source is configured to generate the illumination light beam. The optical sensor is configured to generate a data signal in response to receiving the back-reflected illumination light beam from the laser probe. The controller is configured to generate the control signal for the pulsed laser source, generate a restored back-reflected pulse based on back-reflected pulses in the data signal, and present the restored back-reflected pulse on a display.

### BACKGROUND

The present disclosure relates to surgical systems. More particularly, the present disclosure relates to surgical systems and methods that use lasers to treat patients.

Laser light may be used to treat patients in a wide variety of surgical procedures. For example, laser light may be used to treat a patient’s eye during an ophthalmic surgical procedure, such as cataract surgery, vitrectomy surgery, glaucoma surgery, etc. During cataract surgery, laser light may be used to emulsify the lens in the patient’s eye. During vitrectomy surgery, laser light may be used to cut the vitreous in the patient’s eye. During glaucoma surgery, laser light may be used to create an opening in the trabecular network of the patient’s eye.

### SUMMARY

In certain embodiments, a surgical laser system includes a laser probe and a laser subsystem. The laser probe is configured to emit a treatment light beam, emit an illumination light beam, and receive a back-reflected illumination light beam. The laser subsystem includes a pulsed laser source, a continuous wave laser source, an optical sensor, and a controller. The pulsed laser source is configured to generate the treatment light beam in response to a control signal that includes control pulses. The continuous wave laser source is configured to generate the illumination light beam. The optical sensor is configured to generate a data signal in response to receiving the back-reflected illumination light beam from the laser probe. The data signal includes back-reflected pulses. The controller is coupled to the pulsed laser source, the continuous wave laser source, and the optical sensor, and is configured to generate the control signal for the pulsed laser source, generate a restored back-reflected pulse based on the back-reflected pulses in the data signal and present the restored back-reflected pulse on a display.

### BRIEF DESCRIPTION OF THE DRAWINGS

FIG. 1 illustrates an example surgical laser system for performing a laser-assisted ophthalmic surgical procedure, in accordance with certain embodiments of the present disclosure.

FIG. 2A depicts a data flow and processing diagram for generating a restored back-reflected pulse, in accordance with embodiments of the present disclosure.

FIG. 2B depicts another data flow and processing diagram for generating a restored back-reflected pulse, in accordance with embodiments of the present disclosure.

FIG. 3A depicts a diagram illustrating the relationship between certain signals during a data acquisition window, according to embodiments of the present disclosure.

FIG. 3B depicts a diagram illustrating the acquisition of digitized back-reflected pulses, according to embodiments of the present disclosure.

FIG. 3C depicts a diagram illustrating the detection of the pulse peak value of an average back-reflected pulse, according to embodiments of the present disclosure.

FIG. 3D depicts a diagram illustrating pulse width scanning of the average back-reflected pulse, according to embodiments of the present disclosure.

FIG. 3E depicts a diagram illustrating pulse height scanning of the average back-reflected pulse, according to embodiments of the present disclosure.

FIG. 4A depicts a diagram illustrating a restored back-reflected pulse, according to embodiments of the present disclosure.

FIG. 4B depicts a diagram illustrating an example restored back-reflected pulse, according to embodiments of the present disclosure.

FIG. 4C depicts a diagram illustrating an example restored back-reflected pulse, according to embodiments of the present disclosure.

FIG. 5 depicts a flow diagram describing certain functionality for the surgical laser system depicted in FIG. 1, in accordance with certain embodiments of the present disclosure.

### DETAILED DESCRIPTION

Embodiments of the present disclosure will now be described with reference to the figures, in which like reference numerals refer to like parts throughout.

A surgical system that uses lasers for treatment is also known as a surgical laser system. Generally, a surgical laser system includes a laser subsystem that generates laser light, and may include other subsystems, such as a tool subsystem, a fluidic subsystem, a visualization subsystem, etc. The laser subsystem includes a pulsed laser that is optically coupled to a laser probe. The pulsed laser generates a treatment light beam, and the laser probe delivers the treatment light beam to the patient.

One important operating characteristic of a surgical laser system is the output power that is produced by the pulsed laser. Because the output power of the pulsed laser may be controlled by the surgeon, it is important that the output power remains within manufacturer-specified limits at each power setting. Accordingly, many surgical laser systems may be calibrated prior to the commencement of the surgical procedure to verify that the output power of the pulsed laser is within the manufacturer-specified limits.

For example, a technician/operator may direct the laser probe toward an optical sensor of a laser power meter, select the setting that produces the maximum laser power, activate the pulsed laser, and measure the output power of the pulsed laser detected by the laser power meter. Based on the output power measurements, the technician/operator may adjust the operating parameters of the pulsed laser to ensure that the maximum output power of the pulsed laser is within the manufacturer-specified limits.

Unfortunately, simply measuring the output power of the pulsed laser does not allow for accurately verifying that the surgical laser system is functioning properly. For example, proper functioning of the surgical laser system also depends upon the integrity of the optical path between the laser probe and the pulsed laser. The laser probe includes a handpiece and an optical cable that is coupled to the handpiece and the pulsed laser. If the optical connections are loose, misaligned, etc., or if the optical cable is kinked, deformed, etc., the output power of the pulsed laser that is conveyed to the handpiece of the laser probe may be reduced.

Embodiments of the present disclosure advantageously provide systems and methods for verifying the integrity of surgical laser systems that use lasers to treat patients.

In certain embodiments, a surgical laser system may include an infrared (IR) pulsed laser that generates a treatment light beam in the near IR spectrum in response to a control signal provided by a controller. For example, the control signal may include short, periodic control pulses, and the treatment light beam may include short, periodic IR laser pulses. Each IR laser pulse may have a pulse shape that is defined by the intensity of the IR laser pulse over the duration of the pulse. When the laser probe directs the treatment light beam towards a fluid (such as water, vitreous, etc.), each IR laser pulse causes a transient vapor bubble to be formed in the fluid. The size and lifetime (or duration) of the vapor bubble depends on the energy delivered by the IR laser pulse.

When light in the visible spectrum is directed onto the vapor bubble formed by the IR laser pulse, the vapor bubble reflects a portion of the visible light back towards the source of the visible light. The amount of “back-reflected” visible light depends on the size and duration of the vapor bubble, which depends on the energy delivered by the IR laser pulse. Accordingly, the amount of back-reflected visible light is related to, inter alia, the energy delivered by the IR laser pulse. And, similar to an IR laser pulse, the back-reflected visible light has a nominal pulse shape that may be defined by, inter alia, the intensity of the back-reflected visible light over the duration of the vapor bubble, such as a rectangular-shaped pulse, a square-shaped pulse, a triangular-shaped pulse, etc.

Advantageously, the integrity of the optical path from the IR pulsed laser to the laser probe may be verified using back-reflected visible light that is generated by vapor bubbles produced by IR laser pulses that are directed into a calibration container that is filled with calibration fluid, such as water, etc. More particularly, an optical path that has lost integrity will reduce the output power of the IR pulsed laser that is conveyed to the handpiece of the laser probe. A reduction in the IR pulsed laser output power also reduces the size and duration of the vapor bubbles that are generated in the calibration container by the IR laser pulses, which changes the pulse shape of the back-reflected visible light that is generated by the vapor bubbles. The change in the pulse shape of the back-reflected visible light from the nominal pulse shape may be used to determine that the optical path between the IR pulsed laser and the laser probe has lost integrity.

In certain embodiments, the surgical laser system also includes a continuous wave (CW) laser and an optical sensor that are optically coupled to the laser probe. The CW laser generates an illumination light beam in a portion of the visible spectrum, and the laser probe directs the illumination light beam to the same area as the treatment light beam. Consequently, the illumination light beam illuminates any vapor bubbles formed by the IR laser pulses, which reflect a portion of the illumination light beam back towards the laser probe. The back-reflected illumination light beam is received by the laser probe and conveyed to the optical sensor for measurement.

The optical sensor generates a data signal in response to receiving the back-reflected illumination light beam from the laser probe. The data signal includes back-reflected pulses that correspond to the vapor bubbles generated by the IR laser pulses in the treatment light beam. As discussed above, each back-reflected pulse has a pulse shape that may be defined by, inter alia, the intensity of the back-reflected illumination beam light over the duration of the vapor bubble. The back-reflected pulses have similar pulse shapes when the output power of the IR pulsed laser is within the manufacturer-specified limits and the optical path between the IR pulsed laser and the laser probe has integrity.

The data signal may be processed by the controller to identify one or more of the back-reflected pulses, which may be presented to the technician/operator on a display during calibration. The technician/operator may then visually inspect the back-reflected pulses to determine whether the optical path between the IR pulsed laser and the laser probe has integrity. Unfortunately, the signal level of the back-reflected pulses is low and noisy, which causes the back-reflected pulse shapes to be different and complicates (or renders impracticable) the determination made by the technician/operator.

Advantageously, the back-reflected pulses may be further processed to generate a restored back-reflected pulse that reduces the noise and stabilizes the back-reflected pulse signals. The restored back-reflected pulses allow the technician/operator to determine more precisely whether the optical path between the IR pulsed laser and the laser probe has integrity. The surgical laser system may be calibrated after the integrity of the optical path has been verified. Additionally, the restored back-reflected pulses may be monitored by the technician/operator or the surgeon during the surgical procedure to determine whether the integrity of the optical path has been compromised.

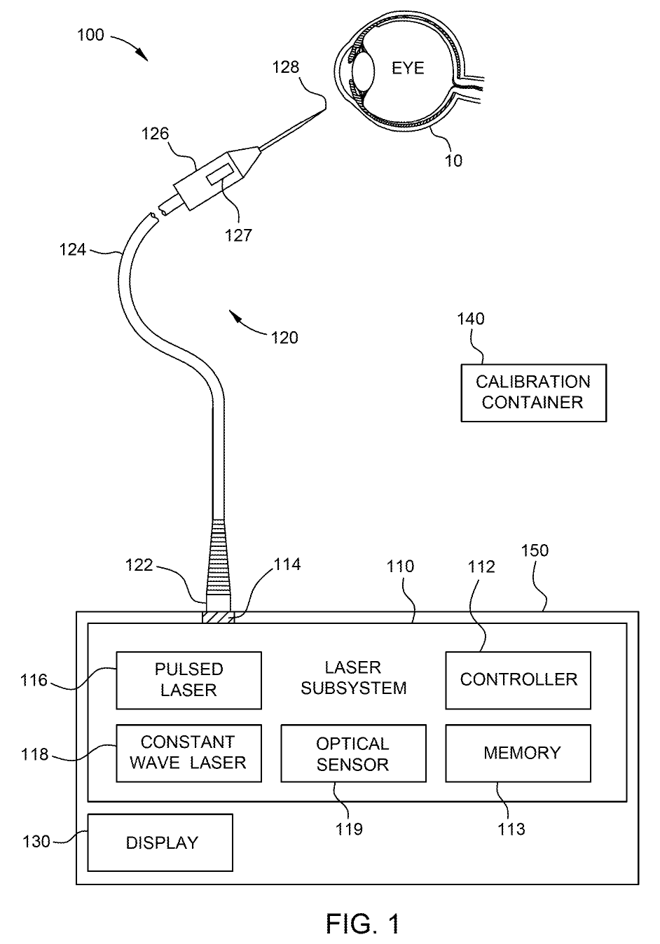

FIG. 1 illustrates an example surgical laser system 100 for performing a laser-assisted ophthalmic surgical procedure on a patient’s eye 10. While described in the context of an ophthalmic surgical procedure, the systems and methods described herein may be used for any suitable medical procedure that uses laser light for treatment purposes, such as tumor removal, wound and scar treatments, dermatology procedures, etc.

In certain embodiments, the surgical laser system 100 includes, inter alia, a laser subsystem 110, a laser probe 120, and a display 130. Generally, the laser probe 120 may be used in a variety of ophthalmic surgical procedures, such as cataract surgery, vitrectomy, glaucoma surgery, etc., and may be attached and detached from the laser subsystem 110. In certain embodiments, the laser probe 120 may be configured to support a particular ophthalmic surgical procedure. The surgical laser system 100 may also include a calibration container 140 for confirming the optical connectivity and functionality of the laser subsystem 110 and the laser probe 120, as described in more detail below.

The laser subsystem 110 may include, inter alia, a controller 112, a memory 113, an optical port 114, an IR pulsed laser source 116, a CW laser source 118, and an optical sensor 119. The optical port 114 is coupled to the IR pulsed laser source 116 using one optical fiber, and to the CW laser source 118 using another optical fiber. The optical sensor 119 is coupled to the same optical fiber as the CW laser source 118 using a fiber coupler, an optical splitter, etc. Generally, the laser subsystem 110 may include one or more optical ports 114, one or more IR pulsed laser sources 116, one or more CW laser sources 118, and one or more optical sensors 119.

The controller 112 is coupled to the memory 113, the IR pulsed laser source 116, the CW laser source 118, the optical sensor 119, and the display 130. The controller 112 may be a microcontroller, a microprocessor, a programmable logic controller (PLC), a field programmable gate array (FPGA), an application specific integrated circuit (ASIC), etc. Generally, the controller 112 is configured to control the operation of the IR pulsed laser source 116 through various settings (such as pulse width or duration, pulse frequency or period, wavelength, power or intensity, spot size, etc.) and to generate a control signal for the IR pulsed laser source 116, to control the operation of the CW laser source 118 through various settings (such as power or intensity, wavelength, etc.) and to generate a control signal for the CW laser source 118, to receive data from the optical sensor 119, and to perform various other functions, such as signal processing, etc.

The IR pulsed laser source 116 is configured to generate laser light that is formed into a laser beam, such as a treatment light beam. In one example, the IR pulsed laser source 116 generates a treatment light beam in the near IR spectrum (e.g., about 750 nm (nanometers) to 1100 nm) in response to the control signal provided by the controller 112. The control signal includes short, periodic control pulses, and the treatment light beam includes short, periodic IR laser pulses, such as 2–5 nanosecond pulses at a 1 kHz (kilohertz) pulse rate. Each IR laser pulse may have a pulse shape that is defined by the intensity of the IR laser pulse over the duration of the pulse, such as a “Gaussian” pulse shape.

The CW laser source 118 is configured to generate light in the visible spectrum (e.g., about 380 nm to 750 nm) that is formed into a laser beam, such as an illumination light beam. In one example, the CW laser source 118 generates an illumination light beam in the red spectrum (e.g., between 620 nm and 750 nm) in response to a control signal provided by the controller 112.

The laser probe 120 includes, inter alia, an optical connector 122, an optical cable 124, and a handpiece 126 including an optical tip 128. The distal end of the optical cable 124 may be directly attached to the handpiece 126; alternatively, the distal end of the optical cable 124 may be removably coupled to the handpiece 126 using an optical connector. The proximal end of optical cable 124 includes the optical connector 122, which may be attached to, and detached from, the optical port 114. One or more optical fibers are located within the optical cable 124, and optically couple the optical connector 122 to the handpiece 126. In one example, the optical cable 124 includes two optical fibers. The first optical fiber conveys the treatment light beam from the optical port 114 to the handpiece 126. The second optical fiber conveys the illumination light beam from the optical port 114 to the handpiece 126 and conveys the back-reflected illumination light beam from the handpiece 126 to the optical port 114.

In certain embodiments, each of the one or more optical fibers may be a single-core optical fiber (SCF) or a multi-core optical fiber (MCF), such as GeO2 with tapped sapphire cable. Other fiber optical materials may also be used, such as Ti:Sapphire, Y3Al5O12 (YAG), Ho:YAG, Yb:YAG, Nd:YAG, Er:YAG, Ce:YAG, Cr:YAG, or ZrF4-BaF2-LaF3-AlF3-NaF (ZBLAN), etc.

The user, such as a surgeon, may activate and deactivate the IR pulsed laser source 116 using a mechanical or electrical control, such as a foot pedal, a switch 127 on the handpiece 126, etc. The mechanical or electrical control may be coupled to the controller 112 or directly to the IR pulsed laser source 116. In one example, an electrical cable may couple the foot pedal to the controller 112. In another example, one or more electrical signal wires located within the optical cable 124 may couple the switch 127 to the optical connector 122, which may include an electrical connector that is configured to attach to a respective electrical connector in the optical port 114.

In certain embodiments, the surgical laser system 100 may include a surgical console 150 that integrates the laser subsystem 110, the display 130, and additional tools and subsystems to facilitate the performance of the ophthalmic surgical procedure. One example of a console configured for performing vitreoretinal procedures is the Constellation® System available from Alcon Laboratories, Inc., Fort Worth, Texas. One example of a console configured for performing cataract surgeries is the Centurion® System available from Alcon Laboratories, Inc., Fort Worth, Texas.

Advantageously, the integrity of the surgical laser system 100 may be verified prior to the commencement of the surgical procedure. More particularly, the technician/operator may verify the integrity of the optical path from the IR pulsed laser source 116 to the laser probe 120 by activating the IR pulsed laser source 116 and the CW laser source 118 and directing the treatment laser beam and the illumination light beam emitted by the laser probe 120 into the calibration container 140, which contains a calibration fluid (such as water). The treatment light beam includes IR laser pulses that produce vapor bubbles in the calibration fluid, which reflect a portion of the illumination light beam back to the laser probe 120 as a back-reflected illumination light beam.

The laser probe 120 receives the back-reflected illumination light beam, which is transmitted through the optical cable 124 and the optical port 114 to the optical sensor 119, which generates a data signal 115 in response to receiving the back-reflected illumination light beam. The data signal 115 includes back-reflected pulses that correspond to the vapor bubbles generated by the IR laser pulses in the treatment light beam. The controller 112 processes the back-reflected pulses within the data signal 115 to generate a restored back-reflected pulse, which is presented to the technician/operator on the display 130. The technician/operator may then visually inspect the back-reflected pulses to determine whether the optical path between the IR pulsed laser source 116 and the laser probe 120 has integrity. As discussed above, the restored back-reflected pulses may also be monitored by the technician/operator or the surgeon during the surgical procedure to determine whether the integrity of the optical path has been compromised.

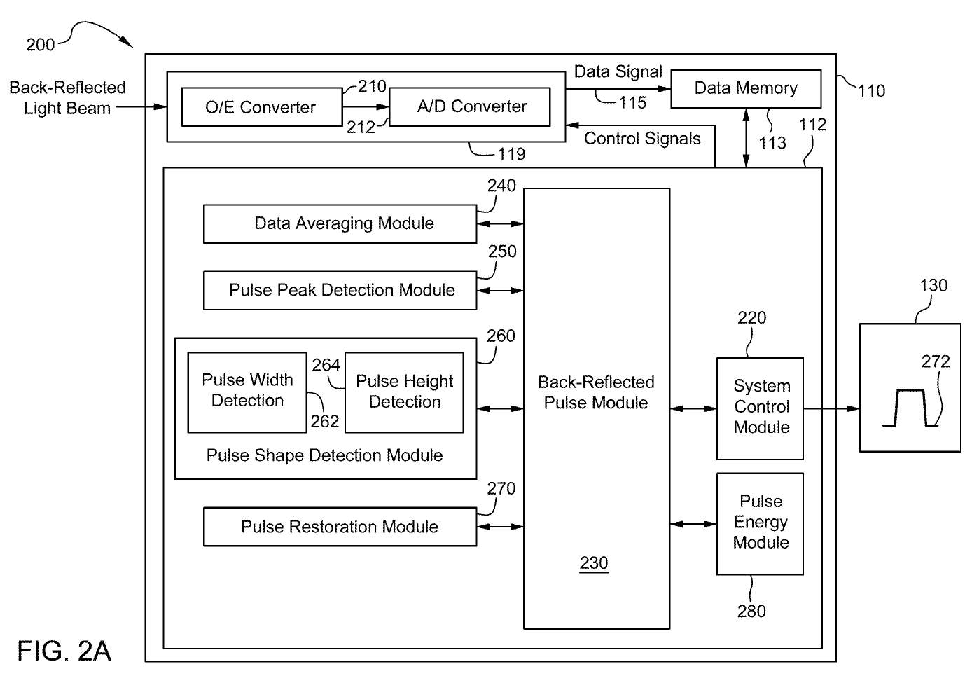

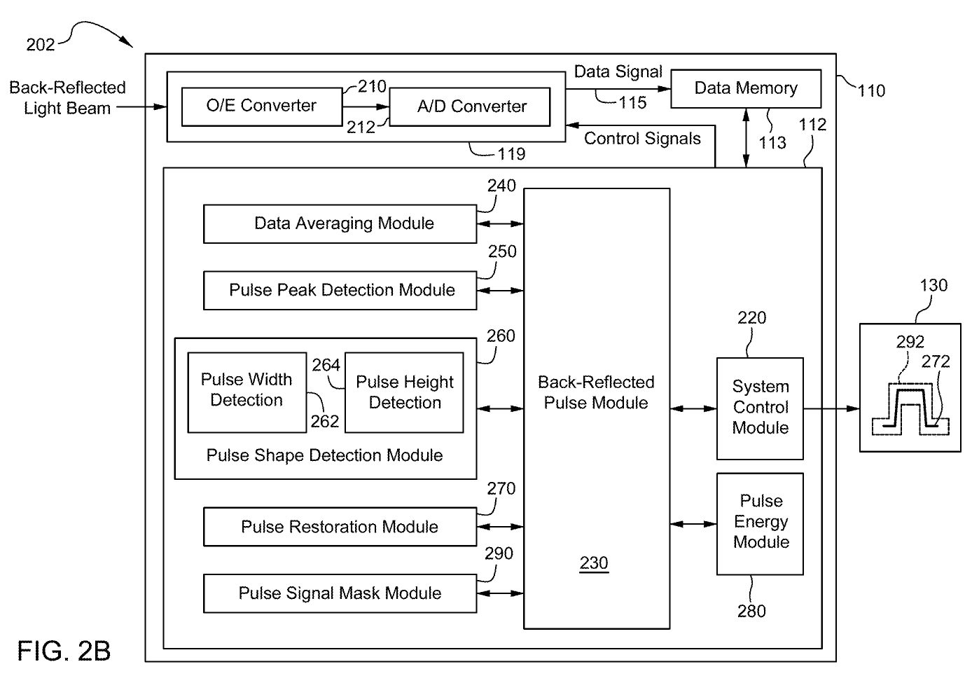

FIG. 2A depicts a data flow and processing diagram 200 for generating a restored back-reflected pulse, in accordance with embodiments of the present disclosure.

In certain embodiments, the optical sensor 119 includes an optical-to-electrical (O/E) converter 210 and an analog-to-digital (A/D) converter 212. The O/E converter 210 may include an optical sensing element coupled to a transimpedance amplifier. The back-reflected illumination light beam is received at the optical sensing element of the O/E converter 210, which outputs an analog photocurrent signal proportional to the incident light. The photocurrent signal is provided to the transimpedance amplifier, which converts the photocurrent signal to an analog voltage signal. The analog voltage signal is provided to the A/D converter 212, which converts the analog voltage signal to a digital data signal. The A/D converter 212 has a resolution in bits (such as 8 bits, 12 bits, 16 bits, etc.) and an input voltage range (such as 0 to 200 millivolts (mV), 0 to 1 V (Volt), etc.). The data signal 115 may be provided to the memory 113 for storage, or to the controller 112 for further processing.

Rather than continuously converting (or digitizing) the analog voltage signal from the O/E converter 210, the controller 112 may command the A/D converter 212 to digitize the analog voltage signal during specific data acquisition windows that correspond to the generation of vapor bubbles by the IR laser pulses. Additionally, while a vapor bubble is generated for each IR laser pulse that impacts the calibration fluid in the calibration container 140, back-reflected light from a contiguous sequence of vapor bubbles does not need to be digitized in order to generate a restored back-reflected pulse.

Instead, the data acquisition windows may occur a slower rate than the vapor bubbles are being generated by the IR laser pulses. For example, when the IR pulsed laser source 116 generates IR laser pulses at a 1 kHz rate, the data acquisition windows may occur at a 50 Hz rate, or one data acquisition window for every 20 IR laser pulses. The duration of the data acquisition window may be longer than the duration of a typical back-reflected pulse within the back-reflected illumination light beam, beginning before a back-reflected pulse and ending after the back-reflected pulse.

In certain embodiments, the controller 112 may send a data acquisition start signal to the A/D converter 212 to start digitizing the analog voltage signal from the O/E converter 210, and then send a data acquisition stop command to the A/D converter 212 to stop digitizing the analog voltage signal from the O/E converter 210. The data acquisition start signal and the data acquisition stop signal define the duration of the data acquisition window. The data acquisition start signal is sent to the A/D converter 212 in response to a back-reflected pulse measurement signal that is generated by the controller 112. The back-reflected pulse measurement signal is based on the control signal for the IR pulsed laser source 116 that is also generated by the controller 112.

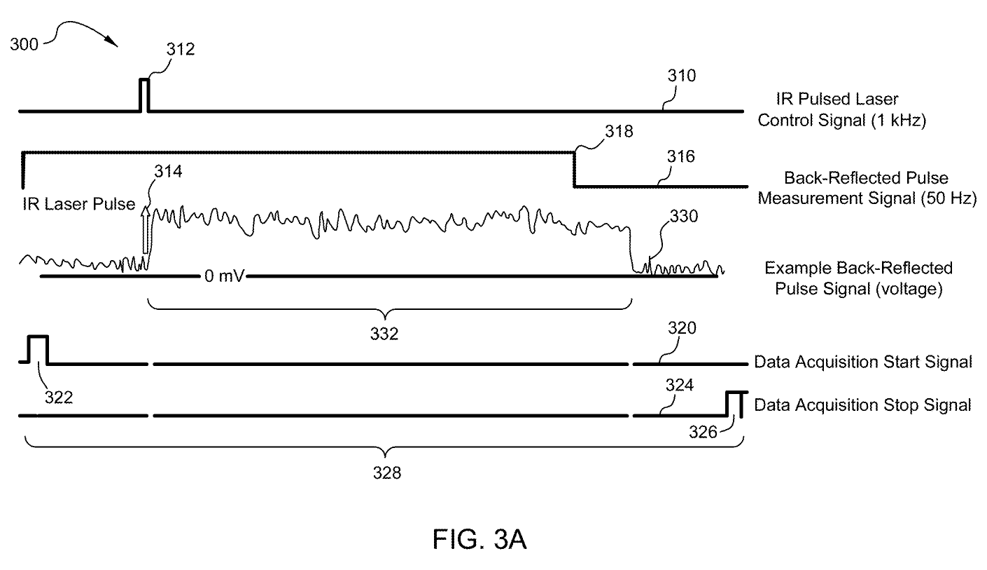

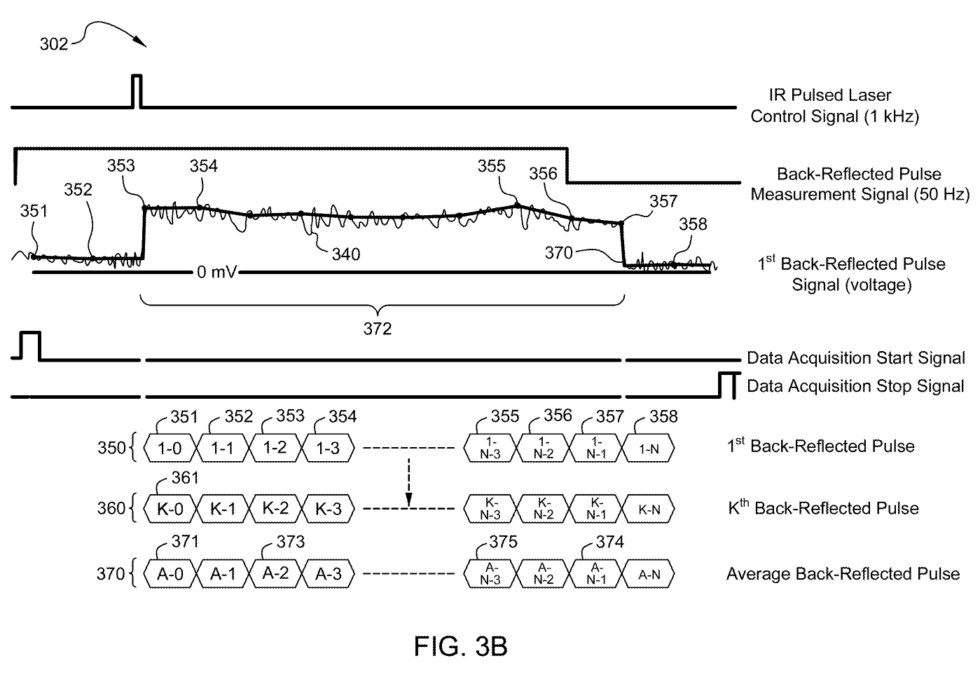

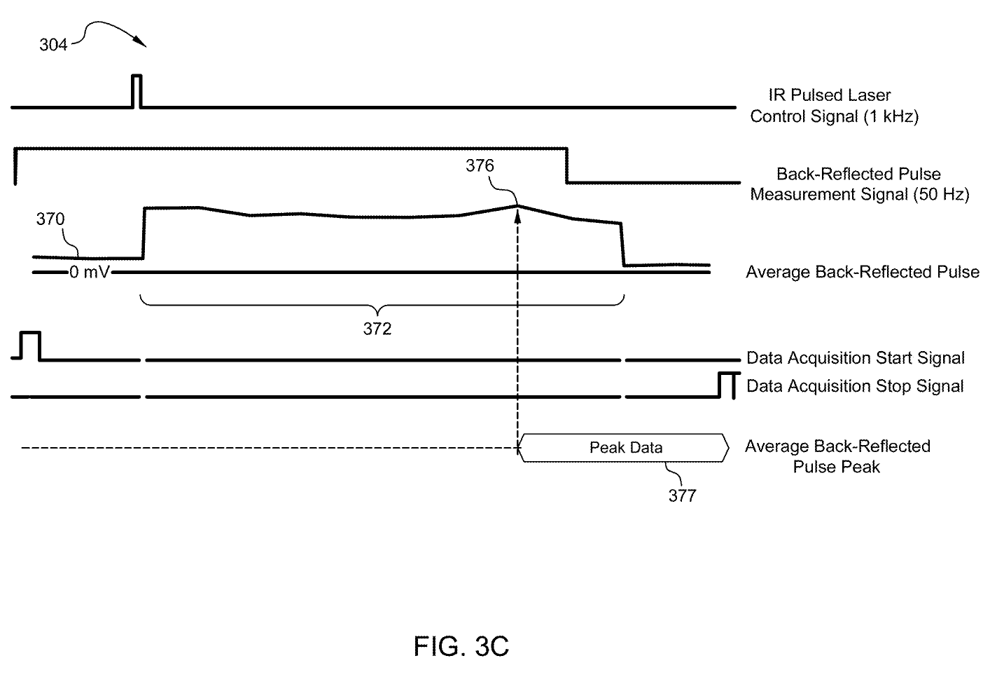

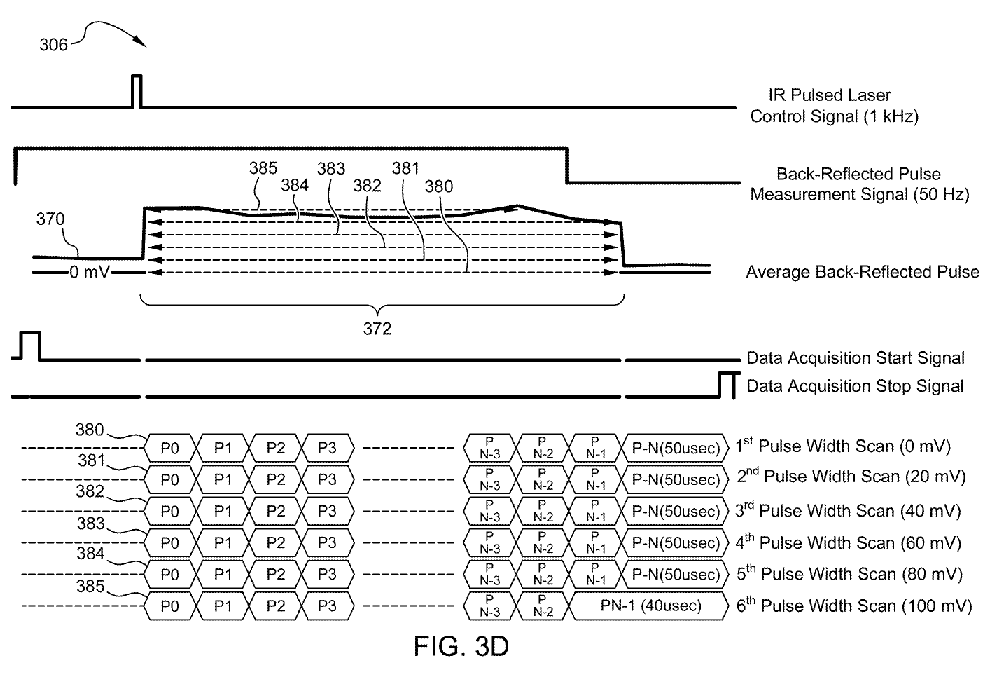

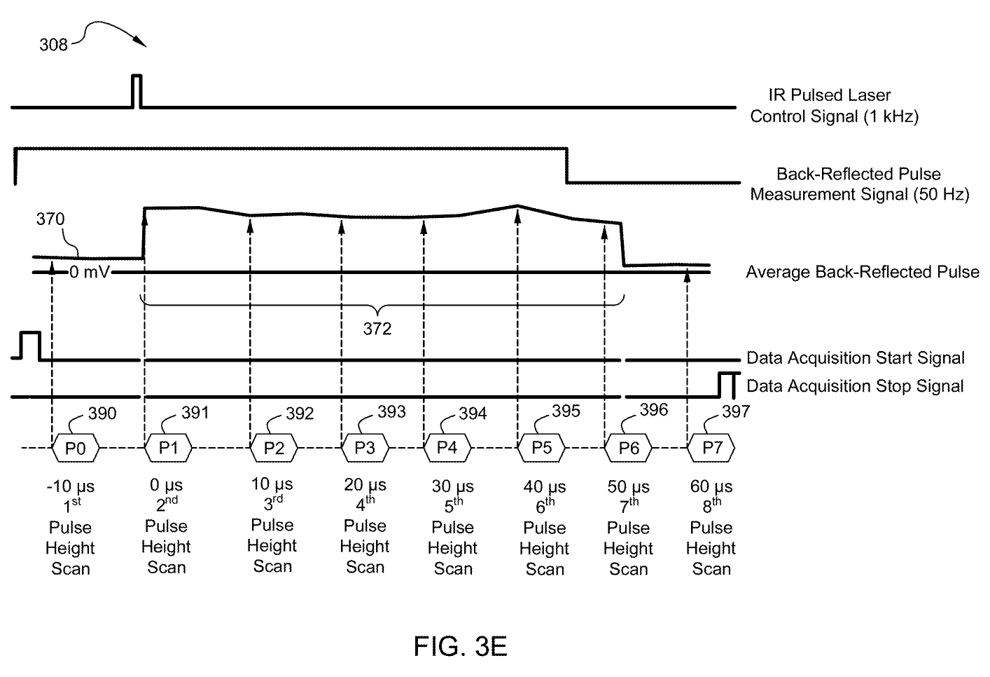

More particularly, the back-reflected pulse measurement signal includes measurement pulses at a particular data acquisition rate (e.g., 50 Hz), and each measurement pulse begins before a control pulse in the control signal for the IR pulsed laser source 116. The data acquisition stop command is sent to the A/D converter 212 after a number of samples (N) have been digitized by the A/D converter 212 during the data acquisition window. The relationship of the measurement pulse in the back-reflected pulse measurement signal, the control pulse in the control signal for the IR pulsed laser source 116, the data acquisition start signal for the data acquisition window, the data acquisition stop command for the data acquisition window, and an example back-reflected pulse analog voltage signal provided by the O/E converter 210 to the A/D converter 212 are depicted in FIG. 3A.

FIG. 3A depicts a diagram 300 illustrating the relationship between certain signals during a data acquisition window 328, according to embodiments of the present disclosure.

The controller 112 generates the control signal 310 for the IR pulsed laser source 116, the back-reflected pulse measurement signal 316, the data acquisition start signal 320 for the A/D converter 212, and the data acquisition stop signal 324 for the A/D converter 212. The control signal 310 includes a control pulse 312, which causes the IR pulsed laser source 116 to generate an IR laser pulse 314 within the treatment light beam. The back-reflected pulse measurement signal 316 starts the data acquisition process for a back-reflected pulse and includes a measurement pulse 318 that begins before the control pulse 312. The data acquisition start signal 320 includes a start pulse 322 that begins after the start of the measurement pulse 318, and the data acquisition stop signal 324 includes a stop pulse 326 that ends after the end of the measurement pulse 318. The start pulse 322 and the stop pulse 326 control the sampling of the back-reflected pulse analog voltage signal 330 by the A/D converter 212.

As discussed above, the IR laser pulse 314 produces a vapor bubble in the calibration fluid of the calibration container 140, which reflects a portion of the illumination light beam back to the laser probe 120 as a back-reflected pulse within the back-reflected illumination light beam. The back-reflected pulse is received at the optical sensing element of the O/E converter 210, which outputs an analog photocurrent signal to the transimpedance amplifier, which converts the photocurrent signal to the example back-reflected pulse analog voltage signal 330, which includes the back-reflected pulse 332. The duration of the data acquisition window 328 begins before the back-reflected pulse 332 and ends after the back-reflected pulse 332.

Referring back to FIG. 2A, in certain embodiments, the controller 112 may execute a number of modules in order to generate a restored back-reflected pulse. The modules may be software modules, firmware modules, a combination of software and firmware modules, etc. The modules may include, inter alia, a system control module 220, a back-reflected pulse control module 230, a data averaging module 240, a pulse peak detection module 250, a pulse shape detection module 260, and a pulse restoration module 270. These modules are discussed in more detail below. In some embodiments, one or more modules may be combined into a single module. For example, the data averaging module 240, the pulse peak detection module 250, the pulse shape detection module 260, and the pulse restoration module 270 may be combined into the back-reflected pulse control module 230. Additionally, the controller 112 may execute a pulse energy module 280 to determine the amount of energy within an IR laser pulse based on the data signal 115 or the restored back-reflected pulses.

Generally, the system control module 220 controls the functionality performed by the controller 112. For example, the system control module 220 may generate a number of signals, such as the control signal 310 for the IR pulsed laser source 116, the control signal for the CW laser source 118, the back-reflected pulse measurement signal 316 for the back-reflected pulse control module 230, etc. The system control module 220 may also process input signals received by the controller 112, such as an input signal from the foot pedal or switch 127 to control the activation of the IR pulsed laser source 116, an input signal to control the activation of the CW laser source 118, etc. The system control module 220 may also command the back-reflected pulse control module 230 to begin generating the restored back-reflected pulses. The system control module 220 (or another module) may also generate a graphical user interface (GUI) for presentation on the display 130. The GUI may include a depiction of a restored back-reflected pulse generated by the back-reflected pulse control module 230.

The back-reflected pulse control module 230 controls the generation of the restored back-reflected pulses using the data averaging module 240, the pulse peak detection module 250, the pulse shape detection module 260, and the pulse restoration module 270. The back-reflected pulse control module 230 may also generate the data acquisition start and end signals for the data acquisition window. In certain embodiments, the system control module 220 may send a start command to the back-reflected pulse control module 230 to generate a restored back-reflected pulse. In response to receiving the start command from the system control module 220, the back-reflected pulse control module 230 begins the generation of a restored back-reflected pulse by acquiring a number (K) of digitized back-reflected pulses, and then averaging the digitized back-reflected pulses using the data averaging module 240.

FIG. 3B depicts a diagram 302 illustrating the acquisition of digitized back-reflected pulses, according to embodiments of the present disclosure.

In response to receiving a first back-reflected pulse measurement signal 316 from the system control module 220, the back-reflected pulse control module 230 generates and provides a first data acquisition start signal 320 to the A/D converter 212. In response to receiving the start pulse 322 of the first data acquisition start signal 320, the A/D converter 212 begins digitizing the first back-reflected pulse analog voltage signal 340 provided by the O/E converter 210 to generate the first back-reflected pulse 350. The A/D converter 212 generates N samples and provides them to the memory 113 for storage, such as the first sample 351, the second sample 352, the third sample 353, the fourth sample 354, …, the N−3 sample 355, the N−2 sample 356, the N−1 sample 357, and the N^{th} sample 358. The back-reflected pulse control module 230 then generates and provides a first data acquisition stop signal 324 to the A/D converter 212. In response to receiving the stop pulse 326 of the first data acquisition stop signal 324, the A/D converter 212 stops digitizing the first back-reflected pulse analog voltage signal 340 provided by the O/E converter 210.

In response to receiving a second back-reflected pulse measurement signal 316 from the system control module 220, the back-reflected pulse control module 230 generates and provides a second data acquisition start signal 320 to the A/D converter 212. In response to receiving the start pulse 322 of the second data acquisition start signal 320, the A/D converter 212 begins digitizing a second back-reflected pulse analog voltage signal provided by the O/E converter 210 to generate a second back-reflected pulse. The A/D converter 212 generates N samples and provides them to the memory 113 for storage. The back-reflected pulse control module 230 then generates and provides a second data acquisition stop signal 324 to the A/D converter 212. In response to receiving the stop pulse 326 of the second data acquisition stop signal 324, the A/D converter 212 stops digitizing the second back-reflected pulse analog voltage signal provided by the O/E converter 210.

Generally, the back-reflected pulse control module 230 continues to generate data acquisition start signals 320 and data acquisition stop signals 324 for the A/D converter 212 (in response to back-reflected pulse measurement signals 316) until a number of (K) back-reflected pulses have been generated and stored in the memory 113, e.g., the first back-reflected pulse 350, …, the K^{th} back-reflected pulse 360. The number (K) of back-reflected pulses may be 2, 4, 8, 16, 32, 64, etc.

The back-reflected pulse control module 230 then sends a command to the data averaging module 240 to generate an average back-reflected pulse 370 by averaging the individual samples of the first back-reflected pulse 350 through the K^{th} back-reflected pulse 360. For example, the first sample 351 of the first back-reflected pulse 350, …, the first sample 361 of the K^{th} back-reflected pulse 360 are summed together and then divided by K to generate the first average sample value 371 of the average back-reflected pulse 370, and so on for the remaining average sample values. The average back-reflected pulse 370 is then stored in the memory 113 for further processing.

The average back-reflected pulse 370 includes a back-reflected pulse 372 beginning at the third average sample value 373 of the average back-reflected pulse 370, and ending at the 12^{th} average sample value 374 of the average back-reflected pulse 370. The 10^{th} average sample value 375 is also identified, which is the peak of the average back-reflected pulse 370. The 10^{th} average sample value 375 is also the pulse peak value 376 (depicted in FIG. 3C).

Referring back to FIG. 2A, the back-reflected pulse control module 230 then sends a command to the pulse peak detection module to 250 to determine the peak of the average back-reflected pulse 370, which returns the pulse peak value to the back-reflected pulse control module 230 (such as 80 mV, 100 mV, 120 mV, etc.). For example, the pulse peak detection module to 250 may simply examine the values of the average back-reflected pulse 370 to find the maximum value, which is provided as the pulse peak value 376 to the back-reflected pulse control module 230.

FIG. 3C depicts a diagram 304 illustrating the detection of the pulse peak value 377 of the average back-reflected pulse 370, according to embodiments of the present disclosure.

The pulse peak detection module 250 may also determine a number of pulse width scans (N_{pw}) to be performed by the pulse width detection module 262 based on the pulse peak value (such as 3 vertical scans, 5 vertical scans, 7 vertical scans, etc.). The number of pulse width scans (N_{pw}) may also be known as a vertical scan number. The pulse width scans are described in more detail below. In some embodiments, the pulse peak detection module to 250 may detect the peak of each back-reflected pulse 350, …, 360, determine the largest pulse peak, and then return the largest pulse peak value to the back-reflected pulse control module 230. In other embodiments, the pulse peak detection module to 250 may detect the peak of a single back-reflected pulse, such as the back-reflected pulse 350, and then return the pulse peak value to the back-reflected pulse control module 230.

Referring back to FIG. 2A, the back-reflected pulse control module 230 then sends a command, including the pulse peak value 376, to the pulse shape detection module 260. Generally, the pulse shape detection module 260 determines certain shape-related characteristics of the average back-reflected pulse 370. In certain embodiments, the pulse shape detection module 260 sends a command to the pulse width detection module 262 to scan the average back-reflected pulse 370 in a horizontal direction, and then sends a command to the pulse height detection module 264 to scan the average back-reflected pulse 370 in a vertical direction. In other embodiments, the pulse shape detection module 260 may first send the command to the pulse height detection module 264, and then send the command to the pulse width detection module 262. In some embodiments, the pulse shape detection module 260 includes the functionality of the pulse width detection module 262 and the pulse height detection module 264, and no commands are needed.

The pulse shape detection module 260 may send a command to the pulse width detection module 262 to perform N_{pw} pulse width scans on the average back-reflected pulse 370. The pulse width scans are performed at different voltage levels that are evenly spaced between 0 mV and the pulse peak value 376, and each pulse width scan provides the width of the average back-reflected pulse 370 in microseconds (μs) at a particular voltage level.

FIG. 3D depicts a diagram 306 illustrating pulse width scanning of the average back-reflected pulse 370, according to embodiments of the present disclosure.

In this example, the pulse peak value 376 is equal to 100 mV and the N_{pw} is equal to 5, so 5 pulse width scans may be performed at 20 mV, 40 mV, 60 mV, 80 mV, and 100 mV. A baseline pulse width scan at 0 mV is also performed, for a total of 6 pulse width scans, e.g., pulse width scan 380, pulse width scan 381, pulse width scan 382, pulse width scan 383, pulse width scan 384, and pulse width scan 385.

The 1^{st} pulse width scan 380 is performed at 0 mV and starts at the beginning of the back-reflected pulse 372 (such as the third average sample value 373 of the average back-reflected pulse 370), and continues until the end of the back-reflected pulse 372 (such as the 12^{th} average sample value 374 of the average back-reflected pulse 370). Because each average sample value has a value greater than 0 mV, the 1^{st} pulse width scan 380 has a time value of 50 μs (i.e., the time difference between the third average sample value 373 and the 12^{th} average sample value 374 of the average back-reflected pulse 370).

The 2^{nd} pulse width scan 381 is performed at 20 mV and starts at the beginning of the back-reflected pulse 372 (such as the third average sample value 373 of the average back-reflected pulse 370), and continues until the end of the back-reflected pulse 372 (such as the 12^{th} average sample value 374 of the average back-reflected pulse 370). Because each average sample value has a value greater than 20 mV, the 2^{nd} pulse width scan 381 has a time value of 50 μs (i.e., the time difference between the third average sample value 373 and the 12^{th} average sample value 374 of the average back-reflected pulse 370).

The 3^{rd} pulse width scan 382 is performed at 40 mV and starts at the beginning of the back-reflected pulse 372 (such as the third average sample value 373 of the average back-reflected pulse 370), and continues until the end of the back-reflected pulse 372 (such as the 12^{th} average sample value 374 of the average back-reflected pulse 370). Because each average sample value has a value greater than 0 mV, the 3^{rd} pulse width scan 382 has a time value of 50 μs (microseconds) (i.e., the time difference between the third average sample value 373 and the 12^{th} average sample value 374 of the average back-reflected pulse 370).

The 4^{th} pulse width scan 383 is performed at 60 mV and starts at the beginning of the back-reflected pulse 372 (such as the third average sample value 373 of the average back-reflected pulse 370), and continues until the end of the back-reflected pulse 372 (such as the 12^{th} average sample value 374 of the average back-reflected pulse 370). Because each average sample value has a value greater than 0 mV, the 4^{th} pulse width scan 383 has a time value of 50 μs (i.e., the time difference between the third average sample value 373 and the 12^{th} average sample value 374 of the average back-reflected pulse 370).

The 5^{th} pulse width scan 384 is performed at 80 mV and starts at the beginning of the back-reflected pulse 372 (such as the third average sample value 373 of the average back-reflected pulse 370), and continues until the end of the back-reflected pulse 372 (such as the 12^{th} average sample value 374 of the average back-reflected pulse 370). Because each average sample value has a value greater than 0 mV, the 5^{th} pulse width scan 384 has a time value of 50 µs (i.e., the time difference between the third average sample value 373 and the 12^{th} average sample value 374 of the average back-reflected pulse 370).

The 6^{th} pulse width scan 385 is performed at 100 mV and starts at the beginning of the back-reflected pulse 372 (such as the third average sample value 373 of the average back-reflected pulse 370), and continues until the peak of the back-reflected pulse 372 (such as the 10^{th} average sample value 375 of the average back-reflected pulse 370). Because at least the third average sample value 373 and the 10^{th} average sample value 375 have a value greater than 100 mV, the 6^{th} pulse width scan 385 has a time value of 40 μs (i.e., the time difference between the third average sample value 373 and the 10^{th} average sample value 375 of the average back-reflected pulse 370).

The time values of the pulse width scans 380, 381, 382, 383, 384, 385 (e.g., 50 μs, 50 μs, 50 μs, 50 μs, 50 μs, and 40 μs) and the associated voltage values (e.g., 0 mV, 20 mV, 40 mV, 60 mV, 80 mV, and 100 mV) may be stored in the memory 113 for further processing.

Referring back to FIG. 2A, the pulse shape detection module 260 may send a command to the pulse height detection module 264 to perform a number of pulse height scans (Nₚₕ) on the average back-reflected pulse 370. The pulse height scans are performed at evenly-spaced times relative to the beginning of the average back-reflected pulse 370, such as every 10 microseconds (μs), every 20 μs, etc. The spacing of the pulse height scans may also be known as the horizontal scan period. Each pulse height scan provides the voltage value of the average back-reflected pulse 370 (in mV) at a particular time (in μs).

FIG. 3E depicts a diagram 308 illustrating pulse height scanning of the average back-reflected pulse 370, according to embodiments of the present disclosure.

In this example, Nₚₕ is equal to 8, so eight pulse height scans are performed. The first pulse height scan 390 may be performed 10 μs before the beginning of the back-reflected pulse 372 (e.g., −10 μs). The second pulse height scan 391 may be performed at the beginning of the back-reflected pulse 372 (e.g., 0 μs). The third pulse height scan 392 may be performed 10 μs after the beginning of the back-reflected pulse 372 (e.g., +10 μs). The fourth pulse height scan 393 may be performed 20 μs after the beginning of the back-reflected pulse 372 (e.g., +20 μs). The fifth pulse height scan 394 may be performed 30 μs after the beginning of the back-reflected pulse 372 (e.g., +30 μs). The sixth pulse height scan 395 may be performed 40 μs after the beginning of the back-reflected pulse 372 (e.g., +40 μs). The seventh pulse height scan 396 may be performed 50 μs after the beginning of the back-reflected pulse 372 (e.g., +50 μs). The eighth pulse height scan 397 may be performed 60 μs after the beginning of the back-reflected pulse 372 (e.g., +60 µs).

The voltage values of the pulse height scans 390, 391, 392, 393, 394, 395, 396, 397 (e.g., 20 mV, 100 mV, 95 mV, 90 mV, 85 mV, 98 mV, 80 mV, and 15 mV) and the associated time values (e.g., −10 μs, 0 μs, +10 μs, +20 μs, +30 μs, +40 μs, +50 μs, and +60 μs) may be stored in the memory 113 for further processing.

Referring back to FIG. 2A, the back-reflected pulse control module 230 then sends a command to the pulse restoration module 270 to generate a restored back-reflected pulse 272. In response, the pulse restoration module 270 generates the restored back-reflected pulse 272 based on the pulse width scan data and the pulse height scan data stored in the memory 113. The restored back-reflected pulse may then be stored in the memory 113.

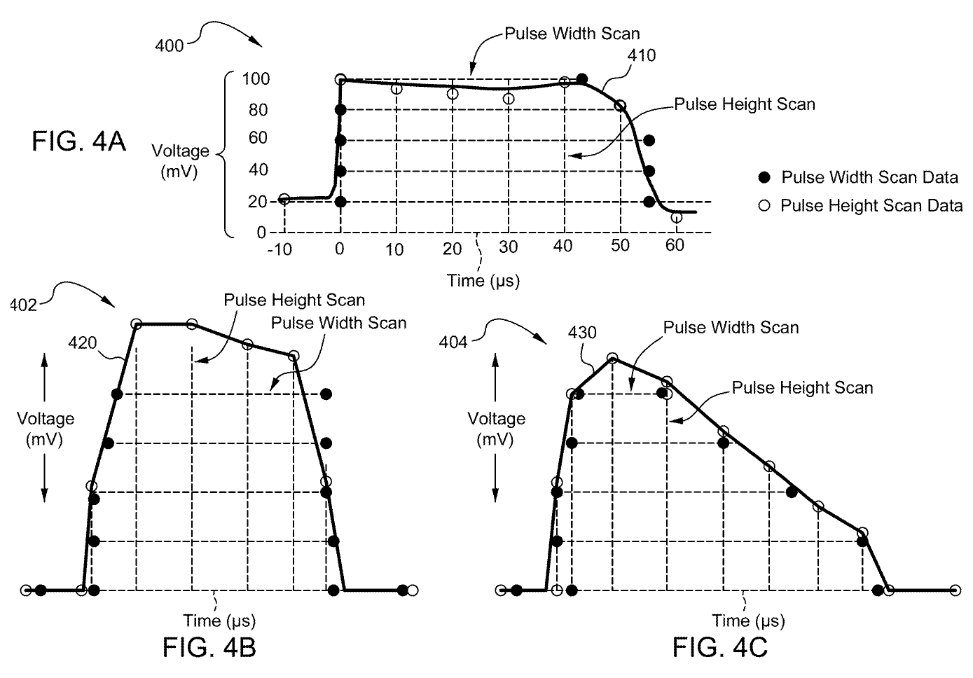

FIG. 4A depicts a diagram 400 illustrating a restored back-reflected pulse 410, according to embodiments of the present disclosure.

In certain embodiments, the restored back-reflected pulse 410 may be generated by interpolating the time values and the voltage values of the pulse width scans 381, 382, 383, 384, 385 (FIG. 3D), and the time values and the voltage values of the pulse height scans 390, 391, 392, 393, 394, 395, 396, 397 (FIG. 3E). In other embodiments, the restored back-reflected pulse 410 may be generated by fitting a curve to the time values and the voltage values of the pulse width scans 381, 382, 383, 384, 385, and the time values and the voltage values of the pulse height scans 390, 391, 392, 393, 394, 395, 396, 397. The time and voltage values for the pulse width scan 380 (0 mV) are not included in the curve fitting data. The restored back-reflected pulse 410 has a generally rectangular-shaped pulse.

FIG. 4B depicts a diagram 402 illustrating an example restored back-reflected pulse 420, according to embodiments of the present disclosure. The restored back-reflected pulse 420 has a generally square-shaped pulse.

FIG. 4C depicts a diagram 404 illustrating an example restored back-reflected pulse 430, according to embodiments of the present disclosure. The restored back-reflected pulse 430 has a generally triangular-shaped pulse.

Referring back to FIG. 2A, the back-reflected pulse control module 230 then notifies the system control module 220 that a new restored back-reflected pulse is available in the memory 113. The system control module 220 provides the restored back-reflected pulse 272 to the display 130 for presentation to the technician/operator.

FIG. 2B depicts another data flow and processing diagram 202 for generating a restored back-reflected pulse, in accordance with embodiments of the present disclosure.

Generally, the data flow and processing diagram 202 adds a pulse signal mask module 290 to the components of the data flow and processing diagram 200. The pulse signal mask module 290 assists the technician/operator in determining whether the optical path between the IR pulsed laser and the laser probe has integrity by comparing the restored back-reflected pulse 272 to a pulse signal mask 292, and then providing an integrity indication to the system control module 220 for presentation to the technician/operator on the display 130.

In certain embodiments, after the pulse restoration module 270 generates the restored back-reflected pulse 272, the back-reflected pulse control module 230 then sends a command to the pulse signal mask module 290 to determine whether the restored back-reflected pulse 272 falls within the boundaries defined by the pulse signal mask 292. The pulse signal mask 292 adds a tolerance to the voltage values of a typical restored back-reflected pulse, such as ± 10%, and may depend on the output power of the IR pulsed laser source 116. When the restored back-reflected pulse 272 falls within the boundaries of the pulse signal mask 292, the pulse signal mask module 290 generates a positive integrity indication. Conversely, when a portion of the restored back-reflected pulse 272 falls outside the boundaries of the pulse signal mask 292 (such as 5%, 10%, etc.), the pulse signal mask module 290 generates a negative integrity indication.

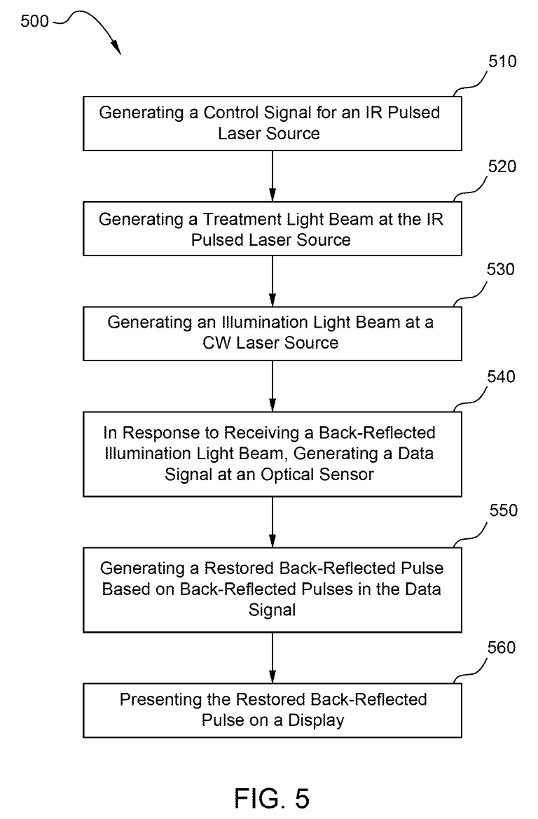

FIG. 5 depicts a flow diagram 500 describing certain functionality for the surgical laser system 100, in accordance with certain embodiments of the present disclosure.

At block 510, the controller 112 generates a control signal for the IR pulsed laser source 116 (e.g., the control signal 310), as described above with respect to FIGS. 2A, 3A. The control signal includes control pulses (e.g., the control pulse 312), which causes the IR pulsed laser source 116 to generate IR laser pulses within the treatment light beam (e.g., the IR laser pulse 314).

At block 520, the IR pulsed laser source 116 generates a treatment light beam in response to the control signal generated by the controller 112.

At block 530, the CW laser source 118 generates an illumination light beam in response to another control signal that is generated by the controller 112, as described above with respect to FIG. 2A.

At block 540, the optical sensor 119 generates a data signal 115 in response to receiving a back-reflected illumination light beam, as discussed above with respect to FIGS. 2A, 3A, 3B. The data signal 115 includes back-reflected pulses that correspond to the vapor bubbles generated by the IR laser pulses in the treatment light beam (e.g., back-reflected pulses 350, 360, etc.).

At block 550, a restored back-reflected pulse 272 is generated based on the back-reflected pulses in the data signal 115, as discussed above with respect to FIGS. 2A, 3B, 3C, 3D, 3E, 4A

At block 560, the restored back-reflected pulse 272 is presented on the display 130, as discussed above with respect to FIG. 2A.

### Example Embodiments

In certain embodiments, a surgical laser system includes a laser probe and a laser subsystem. The laser probe is configured to emit a treatment light beam, emit an illumination light beam, and receive a back-reflected illumination light beam. The laser subsystem includes a pulsed laser source, a continuous wave laser source, an optical sensor, and a controller. The pulsed laser source is configured to generate the treatment light beam in response to a control signal that includes control pulses. The continuous wave laser source is configured to generate the illumination light beam. The optical sensor is configured to generate a data signal in response to receiving the back-reflected illumination light beam from the laser probe. The data signal includes back-reflected pulses. The controller is coupled to the pulsed laser source, the continuous wave laser source, and the optical sensor, and is configured to generate the control signal for the pulsed laser source, generate a restored back-reflected pulse based on the back-reflected pulses in the data signal, and present the restored back-reflected pulse on a display.

In certain embodiments, a method for a surgical laser system comprises generating, at a controller, a control signal for a pulsed laser source, the control signal comprising control pulses; generating, at the pulsed laser source, a treatment light beam in response to the control signal; generating, at a continuous wave laser source, an illumination light beam; generating, at an optical sensor, a data signal in response to receiving a back-reflected illumination light beam, the data signal comprising back-reflected pulses; generating, at the controller, a restored back-reflected pulse based on the back-reflected pulses in the data signal; and presenting, on a display, the restored back-reflected pulse.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A surgical laser system, comprising:
a laser probe configured to:
emit a treatment light beam,
emit an illumination light beam, and
receive a back-reflected illumination light beam; and
a laser subsystem optically coupled to the laser probe, the laser subsystem comprising:
a pulsed laser source configured to generate the treatment light beam in response to a control signal comprising control pulses,
a continuous wave laser source configured to generate the illumination light beam,
an optical sensor configured to generate a data signal in response to receiving the back-reflected illumination light beam from the laser probe, the data signal comprising back-reflected pulses, and
a controller coupled to the pulsed laser source, the continuous wave laser source, and the optical sensor, the controller configured to:
generate the control signal for the pulsed laser source,
generate a restored back-reflected pulse based on the back-reflected pulses in the data signal, and
present the restored back-reflected pulse on a display.

2. The surgical laser system of claim 1, wherein to generate the restored back-reflected pulse comprises:
average a number of back-reflected pulses to generate an average back-reflected pulse.

3. The surgical laser system of claim 2, wherein to generate the restored back-reflected pulse further comprises:
determine a pulse peak in the average back-reflected pulse, the pulse peak having a voltage value;
perform a plurality of pulse width scans on the average back-reflected pulse, each pulse width scan associated with a different voltage value, and each pulse width scan having a time value;
perform a plurality of pulse height scans on the average back-reflected pulse, each pulse height scan associated with a different time value, and each pulse height scan having a voltage value; and
generate the restored back-reflected pulse based on the time values and the voltage values of the pulse width scans, and the time values and the voltage values of the pulse height scans.

4. The surgical laser system of claim 3, wherein:
the plurality of pulse width scans are based on a vertical scan number; and
the different voltage values are based on the pulse peak voltage value and the vertical scan number.

5. The surgical laser system of claim 4, wherein the vertical scan number is at least 3.

6. The surgical laser system of claim 3, wherein the plurality of pulse height scans are based on a horizontal scan period.

7. The surgical laser system of claim 6, wherein the horizontal scan period is 20 microseconds or less.

8. The surgical laser system of claim 3, wherein to generate the restored back-reflected pulse comprises:
fitting a curve to the time values and the voltage values of the pulse width scans, and the time values and the voltage values of the pulse height scans.

9. The surgical laser system of claim 1, wherein the treatment light beam is an infrared light beam, the illumination light beam is a visible light beam, and the back-reflected illumination light beam is a visible light beam.

10. The surgical laser system of claim 9, wherein:
the laser subsystem further comprises an optical port coupled to the pulsed laser source, the continuous wave laser source, and the optical sensor;
the laser probe comprises a handpiece including an optical tip, and an optical cable coupled to the handpiece;
the optical cable comprises an optical connector configured to be coupled to the optical port of the laser subsystem; and
the optical sensor further comprises:
an optical-to-electrical converter configured to convert the back-reflected illumination light beam into a back-reflected analog signal, and
a digital-to-analog converter configured to convert the back-reflected analog signal into the data signal.

11. A method for a surgical laser system, the method comprising:
generating, at a controller, a control signal for a pulsed laser source, the control signal comprising control pulses;
generating, at the pulsed laser source, a treatment light beam in response to the control signal;
generating, at a continuous wave laser source, an illumination light beam;
generating, at an optical sensor, a data signal in response to receiving a back-reflected illumination light beam, the data signal comprising back-reflected pulses;
generating, at the controller, a restored back-reflected pulse based on the back-reflected pulses in the data signal; and
presenting, on a display, the restored back-reflected pulse.

12. The method of claim 11, further comprising:
emitting, at a laser probe, the treatment light beam toward a calibration fluid;
emitting, at the laser probe, the illumination light beam toward a calibration fluid; and
receiving, at the laser probe, the back-reflected illumination light beam from the calibration fluid,
wherein the treatment light beam is an infrared light beam, the illumination light beam is a visible light beam, and the back-reflected illumination light beam is a visible light beam.

13. The method of claim 11, wherein generating the restored back-reflected pulse comprises:
averaging a number of back reflected pulses to generate an average back reflected pulse;
determining a pulse peak in the average back reflected pulse, the pulse peak having a voltage value;
performing a plurality of pulse width scans on the average back reflected pulse, each pulse width scan associated with a different voltage value, and each pulse width scan having a time value;
performing a plurality of pulse height scans on the average back reflected pulse, each pulse height scan associated with a different time value, and each pulse height scan having a voltage value; and
generating the restored back-reflected pulse based on the time values and the voltage values of the pulse width scans, and the time values and the voltage values of the pulse height scans.

14. The method of claim 13, wherein:
the plurality of pulse width scans are based on a vertical scan number;
the different voltage values are based on the pulse peak voltage value and the vertical scan number; and
the plurality of pulse height scans are based on a horizontal scan period.

15. The method of claim 14, wherein generating the restored back-reflected pulse further comprises:
fitting a curve to the time values and the voltage values of the pulse width scans, and the time values and the voltage values of the pulse height scans.
